(19) United States
(12) Patent Application Publication
(10) Pub. No.: US 2020/0138732 A1
(43) Pub. Date: May 7, 2020

(54) Adhesive Skin Patch with Support Liner and Manufacturing Method of the Same

(71) Applicants: NICHIBAN CO., LTD., Tokyo (JP); SENJU USA, INC., Woodland Hills, CA (US)

(72) Inventors: Takao HIRAOKA, Tokyo (JP); Atsushi MURATA, Tokyo (JP); Koji KAWAHARA, Tokyo (JP); Takahiro OGAWA, Woodland Hills, CA (US); Akiharu ISOWAKI, Woodland Hills, CA (US)

(21) Appl. No.: 16/622,156

(22) PCT Filed: Jun. 22, 2018
(86) PCT No.: PCT/US2018/038929

§ 371 Date: Dec. 12, 2019

Related U.S. Application Data
(63) Continuation of application No. 15/630,984, filed on Jun. 23, 2017, now abandoned.

(51) Int. Cl. A61K 9/70; B32B 38/04

(52) U.S. Cl. CPC A61K 9/7084; B32B 38/04; B32B 2307/748; B32B 2038/042; B32B 2556/00

(57) ABSTRACT

An adhesive skin patch with a support liner comprises at least a strip drug product and a release liner longer than the drug product. The support liner includes a backing film and an adhesive layer; the drug product is quadrilateral with two long sides being straight in parallel and two short sides each elliptical arcs convex outward; a longest interval between the two short sides is 20.0 mm or more and 50.0 mm or less, and an interval between the two long sides is 5.0 mm or more and 37.5 mm or less; and the elliptical arc has: an ellipse major axis of 20.0 mm or more and 50.0 mm or less, an ellipse minor axis of 14.8 mm or more and less than 50.0 mm, and the minor axis of at least 2.0 mm wider than the interval of the two long sides.

### BACKGROUND

### 1. Technical Field

The disclosure relates to an adhesive skin patch with a support liner and a manufacturing method of the same.

### 2. Related Art

Conventionally, there has been proposed various adhesive skin patches for human in a medical and health field. These adhesive skin patches are basically constituted of a plurality of layers, such as an adhesive layer, a backing layer, and a release layer. The adhesive layer is applied onto a skin. The backing layer supports the adhesive layer. The release layer protects the adhesive layer until the adhesive layer is applied on a skin. Configurations of respective layers are examined and selected by variously considering the purpose of use, the application site, the presence/absence of skin irritancy and uncomfortable feeling in applying onto an applied surface (skin), the adherability to the skin, the following capability when the skin expands and contracts and to a bending surface, and similar factor.

Recently, in various kinds of adhesive skin patches in which thinning of layers is advanced considering the following capability to the skin and similar factor, there has been proposed to dispose a carrier film on the backing layer in order to improve an operability when the adhesive skin patch is applied to the skin. For example, there has been proposed an adhesive skin patch (Japanese Registered Utility Model No. 3187266) in which a carrier sheet is brought into close contact with a surface of a flexible base material that includes an adhesive layer; and a bandage material (U.S. Pat. No. 6,495,230) in which a backing film is disposed on a surface of a film (base material) that includes an adhesive layer and a grip strip is attached on the backing film.

In order to prevent peeling (turn-up of the adhesive skin patch peripheral portion) from the skin after applying, there has been also propositions that examine the shape. For example, there has been proposed an adhesive skin patch (Japanese Unexamined Patent Application Publication No. 2010-207571) that has a predetermined ratio between a total length of a curved line portion and a total length of a straight line portion, and a radius R of the curved line portion is determined in an outer shape of a surface of the adhesive skin patch; and an adhesive plaster (Japanese Patent No. 4578601) that has a shape combining a halved elliptical shape with a parallelogram.

As described above, there has been propositions in applying the adhesive skin patch to the skin, such as constituting further thinned thickness of each layer of the adhesive skin patch and disposing an appropriate radius (R) in the shape, from various aspects, such as the adherability and the following capability to the skin, the prevention of the uncomfortable feeling in application and an occurrence of turn-up of the peripheral end portion, and similar factor.

The objects of the disclosure is to provide an adhesive skin patch that has an improved operatability of the adhesive skin patch that is constituted of thin layers, especially to provide an adhesive skin patch that has a reduced variation of a drug product area.

### SUMMARY OF THE INVENTION

The inventors seriously studied in order to solve the above-described problems, and first, examined configurations of an adhesive skin patch that achieve an improved handleability of the adhesive skin patch (easy peeling of a release liner and easy application of the adhesive skin patch). As a result, it was found that employing a configuration in which a support liner is attached on a drug product ensured improving the handleability of the adhesive skin patch, such as easy peeling from the release liner and easy application onto an application site when the adhesive skin patch is used.

The inventors also examined configurations that ensure solving an uncomfortable feeling to a skin in application and preventing an occurrence of turn-up of a peripheral end portion, and examined employing a form of a strip as a shape of the drug product, in detail, a quadrilateral drug product that has two short sides being a pair of elliptical arcs that convex outward having lengths equal to one another.

The inventors found that aligning the drug product and a long side of the support liner in the adhesive skin patch in which the above-described quadrilateral drug product having the elliptical arc and the support liner are combined facilitated positioning onto an attachment site, especially preferable for application to a skin adjacent to a mucous membrane. Furthermore, the inventors found that setting the above-described elliptical arc to a specific size made the drug product that is excellent in uniformity of quality with a reduced variation of drug product area. Thus, the disclosure was completed.

The disclosure relates to the following.

[1] An adhesive skin patch in which a support liner is attached includes at least a drug product and a release liner. The drug product is in a form of a strip. The release liner is longer than the drug product. The support liner includes a backing film and an adhesive layer disposed on a surface of the backing film. The support liner is attached to the drug product. The drug product is a quadrilateral drug product with two long sides and two short sides. The long sides are straight lines of approximately equal lengths in parallel with one another, and the two short sides are a pair of elliptical arcs that are convex outward having lengths equal to one another. A longest interval between the two short sides of the drug product is 20.0 mm or more and 50.0 mm or less and an interval between the two long sides of the drug product is 5.0 mm or more and 37.5 mm or less. The elliptical arc is an arc of an ellipse (however, not a true circle arc) having: an ellipse major axis of 20.0 mm or more and 50.0 mm or less; and an ellipse minor axis of 14.8 mm or more and less than 50.0 mm, and the minor axis of at least 2.0 mm wider than the interval between the two long sides of the drug product.

The disclosure further provides the following embodiments.

[2] The adhesive skin patch in which, in the quadrilateral drug product, the longest interval between the two short sides is 28.0 mm or more and 32.0 mm or less and the interval between the two long sides is 12.0 mm or more and 16.0 mm or less. The elliptical arc is an arc of an ellipse having the ellipse major axis of 28.0 mm or more and 32.0 mm or less and the ellipse minor axis of 23.0 mm or more and 27.0 mm or less.
[3] The adhesive skin includes at least an adhesive tape and a release liner. The adhesive tape is a tape-typed drug product in a form of a strip. The adhesive tape includes a backing film and an adhesive layer disposed on one surface of the backing film. The release liner is attached to the adhesive layer. The release liner is longer than the backing film. The support liner is attached to the backing film.
[4] The adhesive skin patch in which the adhesive tape further includes a carrier film that is brought into close contact with a surface on an opposite side of a side of the adhesive layer of the backing film. The support liner is attached to the carrier film.
[5] The adhesive skin patch in which, the carrier film includes one end portion out of close contact with a surface of the backing film of the adhesive tape and separated from the backing film to form a non-attached end portion.
[6] The adhesive skin patch is an adhesive skin patch for an eyelid.

The disclosure further targets the following.

[7] A manufacturing method of an adhesive skin patch in which a support liner is attached. The adhesive skin patch includes at least a drug product and a release liner. The drug product is in a form of a strip. The release liner is longer than the drug product. The support liner includes a backing film and an adhesive layer disposed on a surface of the backing film. The support liner is attached to the drug product. The drug product is a quadrilateral drug product with two long sides and two short sides. The two long sides are straight lines of approximately equal lengths in parallel with one another, and the two short sides are a pair of elliptical arcs that convex outward having lengths equal to one another. A longest interval between the two short sides is 20.0 mm or more and 50.0 mm or less, and an interval between the two long sides is 5.0 mm or more and 37.5 mm or less. The manufacturing method includes: a step of punching out the drug product into a shape including the elliptical arc having: an ellipse major axis of 20.0 mm or more and 50.0 mm or less; and an ellipse minor axis of 14.8 mm or more and less than 50.0 mm, and the minor axis of at least 2.0 mm wider than the interval between the two long sides of the drug product; a step of attaching a support liner material over a whole surface of the drug product punched out into the shape including the elliptical arc; and a step of obtaining the support liner on which the quadrilateral drug product is attached by punching out the support liner material together with the drug product formed by having the support liner material attached.

The disclosure further provides the following embodiments.

[8] The manufacturing method in which, in the quadrilateral drug product, the longest interval between the two short sides is 28.0 mm or more and 32.0 mm or less, and the interval between the two long sides is 12.0 mm or more and 16.0 mm or less. The step of punching out the drug product into the shape including the elliptical arc is a step of punching out the drug product into the shape including the elliptical arc that is an arc of an ellipse having the ellipse major axis of 28.0 mm or more and 32.0 mm or less and the ellipse minor axis of 23.0 mm or more and 27.0 mm or less.
[9] The manufacturing method in which the step of obtaining the support liner is a step of punching out the support liner material into a quadrilateral having a long side length of 50 mm±5 mm and a short side length of 5.0 mm or more and 37.5 mm or less.
[10] The manufacturing method in which the step of obtaining the support liner is a step of punching out the support liner material together with the drug product formed by having the support liner material attached such that: a center axis in a long side direction of the drug product in the shape including the elliptical arc and a center axis in a long side direction of the quadrilateral of the support liner become parallel; a center axis in a short side direction of the drug product in the shape including the elliptical arc and a center axis in a short side direction of the quadrilateral of the support liner are on an identical straight line; and a distance between a center (center of gravity) in the short side direction of the drug product in the shape including the elliptical arc and a center (center of gravity) in the short side direction of the quadrilateral of the support liner is 1.0 mm or less.

In the adhesive skin patch in which the support liner of the disclosure is attached, aligning the drug product and the long side of the support liner facilitates positioning onto the attachment site, especially preferable for the application to the skin adjacent to the mucous membrane. The drug product in the form of the strip is the quadrilateral drug product having the elliptical arc with the two short sides that convex outward having lengths equal to one another. This reduces the turn-up of the peripheral end portion of the drug product when the adhesive skin patch is applied to the skin as the applied surface, thereby ensuring an excellent adherability to the skin.

The adhesive skin patch in which the support liner of the disclosure is attached can be the adhesive skin patch with the reduced variation of the drug product area by employing the quadrilateral drug product having the specific elliptical arc. Therefore, especially in the drug product containing the active ingredients, such as a pharmacological agent, the adhesive skin patch that has the reduced variation in a content of the pharmacological agent and a constant product quality is provided.

With the manufacturing method of the adhesive skin patch with the support liner of the disclosure, the drug product in the form of the strip can be formed into the shape of the quadrilateral having the elliptical arc with the two short sides that convex outward having lengths equal to one another. The existence of this elliptical arc ensures reducing a stress of the drug product in application when the adhesive skin patch is applied to the skin as the applied surface, thus providing the adhesive skin patch including the drug product with the excellent adherability to the skin.

With the manufacturing method of the disclosure, the variation of the drug product area can be reduced (for example, 1.0% or less) even when the displacement (pitch displacement) between the center of gravity of the support liner and the center of gravity of the drug product is generated when the support liner material and the drug product formed by having the support liner material attached are punched out into the shape of the support liner.

Therefore, in the case of the drug product containing the active ingredients, such as the pharmacological agent, the adhesive skin patch including the drug product with the reduced variation in the content of the pharmacological agent can be manufactured.

### BRIEF DESCRIPTION OF DRAWINGS

FIG. 1 is a plan view illustrating one embodiment of an adhesive skin patch 1 in which a support liner of the disclosure is attached;

FIG. 2 is a sectional drawing illustrating one embodiment of the adhesive skin patch 1 in which the support liner of the disclosure is attached;

FIGS. 3A to 3C are drawings illustrating a manufacturing method of the adhesive skin patch in which the support liner of the disclosure is attached and are schematic diagrams of steps of main part structure illustrating in an order of the steps;

FIG. 4A is a plan view illustrating a shape including an elliptical arc of a drug product applied in the disclosure and FIG. 4B is a plan view illustrating a shape of a quadrilateral of the support liner; and

FIGS. 5A to 5C are drawings illustrating a manufacturing method of an adhesive skin patch in which a support liner manufactured in a working example is attached and are schematic diagrams of steps of main part structure illustrating in an order of the steps.

### DETAILED DESCRIPTION

Adhesive Skin Patch in which Support Liner is Attached

The disclosure targets an adhesive skin patch in which a support liner is attached. In detail, the disclosure is constituted of an adhesive skin patch formed by including at least a support liner, a drug product in a form of a strip, and a release liner that is longer than the drug product.

In the disclosure, the support liner attached to the adhesive skin patch is formed by including a backing film and an adhesive layer disposed on a surface of the backing film. The support liner is attached to the drug product.

The drug product in the form of the strip used in the disclosure (hereinafter simply referred to as the drug product) is not particularly limited. The drug product can include, for example, a tape-typed drug product described later.

In the disclosure, the drug product is a drug product in a quadrilateral with two long sides being straight lines of approximately equal lengths in parallel with one another and two short sides being a pair of elliptical arcs that convex outward having lengths equal to one another.

In further detail, the drug product has the longest interval between the two short sides of 20.0 mm or more and 50.0 mm or less and the interval between the two long sides of 5.0 mm or more and 37.5 mm or less. The elliptical arc is an arc of an ellipse (however, not a true circle arc) fulfilling: the ellipse major axis of 20.0 mm or more and 50.0 mm or less; and the ellipse minor axis of 14.8 mm or more and less than 50.0 mm, the minor axis of at least 2.0 mm wider than the interval between the two long sides of the drug product.

For example, it is preferable that the drug product has the longest interval between the two short sides of 28.0 mm or more and 32.0 mm or less and the interval between the two long sides of 12.0 mm or more and 16.0 mm or less. The elliptical arc is preferred to be an arc of an ellipse fulfilling the ellipse major axis of 28.0 mm or more and 32.0 mm or less and the ellipse minor axis of 23.0 mm or more and 27.0 mm or less.

Manufacturing Method of Adhesive Skin Patch in which Support Liner is Attached

The adhesive skin patch in which the support liner of the disclosure is attached is manufactured including at least the following steps.

1) a step of punching out the drug product into the shape including the elliptical arc having: the ellipse major axis of 20.0 mm or more and 50.0 mm or less; the ellipse minor axis of 14.8 mm or more and less than 50.0 mm; and the minor axis of at least 2.0 mm wider than the interval between the two long sides of the drug product;
2) a step of attaching a support liner material on a whole surface of the drug product punched out into the shape including the elliptical arc; and
3) a step of obtaining the support liner on which the quadrilateral drug product is attached by punching out the support liner material together with the drug product, which is formed by having the support liner material attached.

For example, in the step of 1), the drug product can be punched out into the shape including the elliptical arc that is the arc of the ellipse fulfilling the ellipse major axis of 28.0 mm or more and 32.0 mm or less and the ellipse minor axis of 23.0 mm or more and 27.0 mm or less.

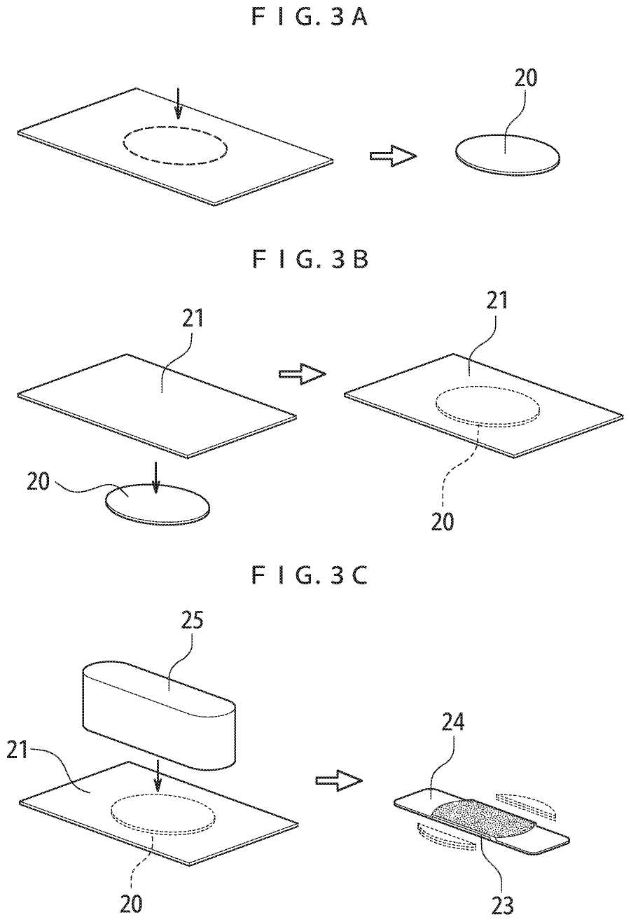

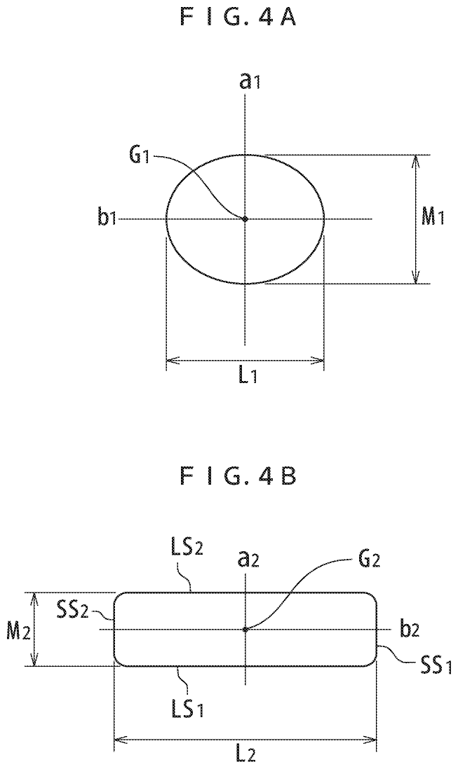

The following describes the above-described steps according to the manufacturing method of the adhesive skin patch of the disclosure in detail with respect to the drawings (see FIGS. 3A to 4B).

1) Step of Punching Out Drug Product into Shape Including Elliptical Arc

This step is a step of punching out the drug product into the shape including the elliptical arc (FIG. 3A). Here, in the case where the drug product in the form of the strip, which can be consequently obtained, is the tape-typed drug product described later, the drug product means the laminated body (adhesive tape) including the backing film and the adhesive layer. In the case where a carrier film is additionally disposed, the drug product means the laminated body (adhesive tape) including the carrier film, the backing film, and the adhesive layer in this order. With this step, a drug product 20 in the shape including the elliptical arc is obtained.

In the above-described shape including the elliptical arc in the disclosure, the elliptical arc is an arc of an ellipse having: an ellipse major axis L₁ of 20.0 mm or more and 50.0 mm or less; an ellipse minor axis M₁ of 14.8 mm or more and less than 50.0 mm; and the minor axis M1 of at least 2.0 mm wider than the interval between the two long sides in the quadrilateral drug product as illustrated in FIG. 4A. As one example of a preferable elliptical arc, an arc of the ellipse fulfilling the ellipse major axis L₁ of 28.0 mm or more and 32.0 mm or less and the ellipse minor axis M₁ of 23.0 mm or more and 27.0 mm or less is included.

In the drug product in the form of the strip, which can be consequently obtained, this step can be said to be a step for, so to say, forming the two short sides in the quadrilateral drug product: a pair of elliptical arcs that convex outward having lengths equal to one another. The ellipse major axis L₁ set here virtually corresponds to the long side of the drug product in the adhesive skin patch of the disclosure.

In this step, while the drug product may be punched out such that the above-described specific elliptical arcs themselves do not overlap, that is, the drug product may be punched out into the elliptical shape, from an aspect of reducing losses of the product, the drug product may be punched out such that a part of the elliptical arc overlaps (that is, the drug product may be punched out into the shape including the elliptical arc).

2) Step of Attaching Support Liner Material to Drug Product Punched Out into Shape Including Elliptical Arc

This step is a step of attaching a support liner material 21 to a whole surface of the drug product 20 punched out into the shape including the elliptical arc (FIG. 3B). Here, the support liner material can be said to be a material that becomes the support liner by punching out in the later step, so to say, a raw material of the support liner.

### 3) Step of Obtaining Support Liner on Which Quadrilateral Drug Product Is Attached

This step is a step of obtaining a support liner 24 on which a quadrilateral drug product 23 is attached by punching out the support liner material 21 into a shape of the support liner (a metallic mold 25) together with the drug product 20 (punched out into the shape including the elliptical arc), which is formed by having the support liner material attached (FIG. 3C).

With this step, the shape of the support liner is formed and the two long sides in the quadrilateral drug product: the straight lines of approximately equal lengths in parallel with one another are formed. With this step, a short side of the support liner becomes virtually identical to the short side of the quadrilateral drug product.

Preferably, in this step, the support liner material is preferred to be punched out, as illustrated in FIG. 4B, into a shape of a quadrilateral support liner having two long sides LS₁ and LS₂ of straight lines of equal lengths in parallel with one another, two short sides SS₁ and SS₂ of straight lines of equal lengths, a long side length L₂ of 50.0 mm±5.0 mm, and a short side length M₂ of 5.0 mm or more and 37.5 mm or less.

In the disclosure, in the shape of the support liner: quadrilateral, the corners may include R. Disposing radii (R) at the respective corners of the support liner can prevent peeling of the support liner from a peeling sheet when the adhesive skin patch in which the support liner of the disclosure is attached is stored.

In a further preferable aspect, it is preferred to punch out into the support liner shape such that: a center axis b₁ in a long side direction of the drug product in the shape including the elliptical arc and a center axis b₂ in a long side direction of the quadrilateral of the support liner are parallel; and a center axis a₁ in a short side direction of the drug product in the shape including the elliptical arc and the center axis a₂ in a short side direction of the quadrilateral of the support liner is on an identical straight line. At this time, it is preferred to punch out such that a distance between a center (center of gravity G₁) in the short side direction of the drug product in the shape including the elliptical arc and a center (center of gravity G₂) in the short side direction of the quadrilateral of the support liner is 1.0 mm or less.

The following describes respective configurations of the drug product in the form of the strip, the release liner, and the support liner. These configurations constitute the adhesive skin patch of the disclosure.

### Drug Product in Form of Strip: Tape-Typed Drug Product

As one aspect of the drug product in the form of the strip used in the disclosure, a form of an adhesive tape that includes the backing film and the adhesive layer disposed on one surface of the backing film can be used. In the case of this embodiment, the release liner described later is attached to the adhesive layer and the support liner described later is attached to the backing film of the adhesive tape. Additionally in this aspect, the carrier film that is brought into close contact with a surface on an opposite side of a side of the adhesive layer of the backing film may be further included. In this case, the support liner described later is attached to the carrier film.

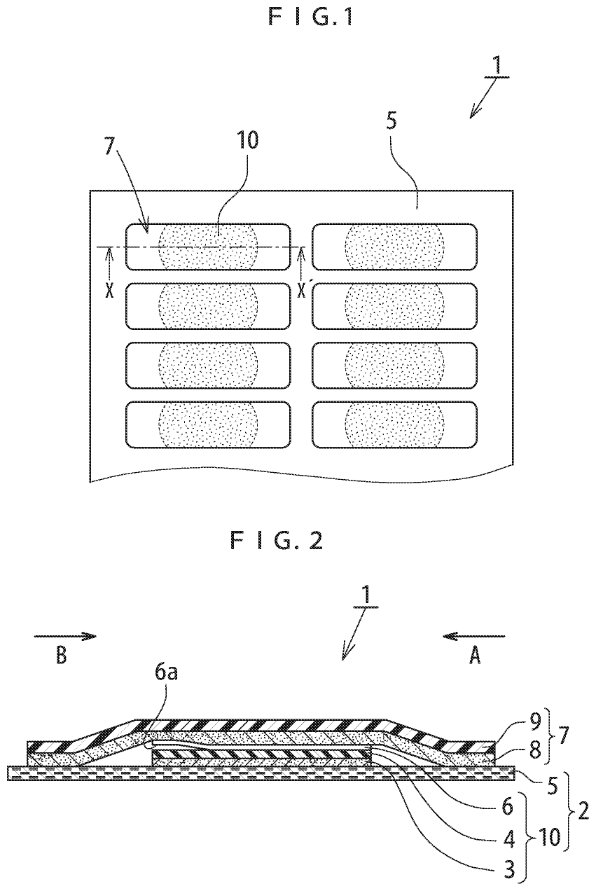

FIG. 1 is a plan view illustrating one preferable embodiment of an adhesive skin patch 1 in which the support liner of the disclosure is attached. As illustrated in FIG. 1, in this embodiment, the adhesive skin patch 1 is constituted of an adhesive tape 10 as the drug product in the form of the strip, a support liner 7 attached to the drug product, and a release liner 5 that is longer than the drug product.

FIG. 2 is a drawing illustrating the most preferable one embodiment of the adhesive skin patch 1 in which the support liner of the disclosure is attached. FIG. 2 illustrates a sectional drawing at a line X-X′ in FIG. 1 described above.

As illustrated in this embodiment the adhesive skin patch 1 in which the support liner is attached is constituted of an adhesive skin patch 2 and the support liner 7 that covers an upper side of the adhesive skin patch 2.

The adhesive skin patch 2 includes the adhesive tape 10 and the release liner 5. The adhesive tape 10 includes a backing film 4 and an adhesive layer 3 disposed on a surface on one side (a lower side in FIG. 2) of the backing film 4. The release liner 5 is attached to the adhesive layer 3 and is longer than the backing film 4. In the preferable aspect, as illustrated in FIG. 2, a carrier film 6 that is brought into close contact with a surface on the opposite side of the adhesive layer 3 side of the backing film 4 in the adhesive tape 10 may be disposed. At this time, a non-attached end portion 6a that is in a state of not being in close contact with the surface of the backing film 4 and being separated from the backing film 4 in an end portion on one side of the carrier film 6 is formed. Forming the non-attached end portion 6a causes the non-attached end portion 6a to serve as an origin to improve easy peeling the carrier film 6 from the backing film 4.

A method for using the adhesive skin patch of the disclosure (a method for applying onto a skin as an applied surface) is, for example, as follows.

First, the support liner 7 is peeled off from the release liner 5 at the opposite side (side A in FIG. 2) of a side where the non-attached end portion 6a of the carrier film 6 is formed. Then, pulling the support liner 7 upward as it is with respect to the release liner 5 subsequently peels off the adhesive tape 10 (the backing film 4, the adhesive layer 3, and, in the case of this aspect, the carrier film 6) from the release liner 5.

Subsequently, the adhesive layer 3 of the adhesive tape 10 is placed applied on the applied surface (skin). At this time, an adhesive layer 8 of the support liner 7 may be temporarily applied to the applied surface (skin).

Then, (in the case where the adhesive layer 8 of the support liner 7 is applied onto the applied surface, the support liner 7 is peeled off from the applied surface, then) pulling the support liner 7 upward with respect to the applied surface at a side (side B in FIG. 2) where the non-attached end portion 6a of the carrier film 6 is formed peels off the carrier film 6, which is formed by attaching to the support liner 7, from the backing film 4 of the adhesive tape 10, and the adhesive tape (the backing film 4 and the adhesive layer 3) are applied onto the applied surface (skin).

Thus, the support liner 7 is used for facilitating peeling of the adhesive tape 10 from the release liner 5 and applying of the adhesive layer 3 of the adhesive tape 10 onto the targeted applied surface. The support liner 7 also plays a role of being used for easily peeling off the carrier film 6 formed by being in close contact with to the backing film 4 of the adhesive tape 10.

### Backing Film

The backing film used for the above-described adhesive tape includes, for example: a paper, such as an impregnated paper, a coated paper, a high-quality paper, a kraft paper, a Japanese paper, and a glassine paper; a plastic film, such as a polyester film, such as polyethylene terephthalate and polybutylene terephthalate, a polyolefin film, such as polyethylene and polypropylene, a polyvinyl chloride film, a polycarbonate film, a polyurethane film, and a cellophane film; a foam; a fabric, such as nonwoven fabric, woven fabric, and knitted fabric made of polyester, polyurethane, polyethylene, and polypropylene; and a laminated body of two or more kinds of these.

Among these, a material flexible enough to come in close contact with the skin and follow a movement of the skin and a material that can inhibit an occurrence of, for example, a skin rash after a long time application is preferred. Among these, the polyester film, the polyurethane film, or the polyolefin film is preferred with the point of excellent followability to the movement (expansion and contraction portion and bending surface) of the skin under the application, and in particular, it is preferred to use the polyolefin film, even more, the polyethylene film.

The backing film can be set to have, for example, its thickness within a range of 5 μm to 1 mm by considering, for example, a physical property, such as a tensile elongation, a tensile strength, and a workability, a touch of application, sealability of an affected part, transferability of each constituent (for example, medical agent) included in the adhesive layer described later to the backing film. The thickness of the backing film is preferred to be 5 μm to 300 μm, more preferably, 10 μm to 100 μm, and yet more preferably, 10 μm to 50 μm. When the thickness of the backing film is smaller (thinner) than 5 μm, a strength and a handleability of the adhesive tape lower to make it difficult to apply onto the skin even though the carrier film is disposed. This possibly causes a contact with, for example, other members, a breakage at the expansion and contraction portion and the bending surface of the skin, and peeling off from the skin in a short time due to a contact with water, such as bathing. When the thickness of the backing film is too large (exceeding 1 mm), the adhesive tape hardly follow the movement of the skin to easily form a start of peeling at a side edge portion of the adhesive tape. Therefore, the adhesive tape may be peeled off from the skin in a short time and an uncomfortable feeling during the application may be increased.

The backing film is preferred to be set to have, for example, its tensile strength within the range of 1 N/15 mm to 100 N/15 mm and its tensile elongation within the range of 50% to 1500% by considering ensuring a flexibility that can follow the expansion and contraction portion and the bending surface of the skin and an adhesion to the skin. When the tensile strength of the backing film is less than 1 N/15 mm, the strength is low, thus making it hard to follow in the expansion and contraction portion and the bending surface of the skin. Meanwhile, when the tensile strength exceeds 100 N/15 mm, the flexibility lacks on the other hand. Also in this case, following in the expansion and contraction portion and the bending surface of the skin, especially following in expansion becomes hard, and additionally, a repeated use may cause a skin irritation due to a large resistance caused in expansion. When the tensile elongation is less than 50%, following in the expansion and contraction portion and the bending surface of the skin lacks. When the tensile elongation exceeds 1500%, while following in the expansion and contraction portion and the bending surface of the skin is sufficient, repeated expansion, contraction and bending cause lack of adhesion onto the skin, therefore quick following becomes insufficient.

On the backing film, a process, such as a sand-blasting process and a corona treatment, may be performed onto a surface where the adhesive layer of the backing film is disposed for an object to improve an anchoring property of the adhesive and the backing film.

Furthermore, in order to make the adhesive tape inconspicuous when being applied to the skin, a film that is high in transparency may be employed or the adhesive tape may be colored into a color tone, such as a skin color, with a colorant, such as pigments, organic pigments, and natural dyes, so as to reduce a mutual difference with the color of the skin when being attached.

The backing film can include additives, such as an antistatic agent and an ultraviolet inhibitor, to an extent that an effect of the disclosure is not hindered. As the antistatic agent, for example, surfactant (anionic surfactant, a cationic surfactant, nonionic surfactant, and amphoteric surfactant) is included. The antistatic agent can solve failures of the anchoring property and the step of the backing film.

### Adhesive Layer

As an adhesive that forms the above-described adhesive layer in the adhesive tape, for example, an acrylic adhesive, a rubber adhesive, an urethane adhesive, and a silicone adhesive can be included. One of these adhesives may be used alone or two or more kinds of these adhesives may be mixed and used. Among these, it is preferable to use the rubber adhesive from the aspect of, for example, a compatibility with compound components.

The rubber adhesive generally includes a rubber elastomer, a tackiness agent, and a softener, and as necessary, various kinds of additives, such as a filler and an antioxidizing agent (antioxidant) described later, are further added.

As the rubber elastomer, various kinds of thermoplastic elastomer is applicable, including: a thermoplastic block copolymer, such as a styrene-isoprene-styrene copolymer (hereinafter possibly referred to as “SIS”), a styrene-butadiene-styrene copolymer (hereinafter possibly referred to as “SBS”), or the hydrogen additives of these, a styrene-ethylene-propylene-styrene copolymer (hereinafter possibly referred to as “SEPS”), a styrene-ethylene-butylene-styrene copolymer (hereinafter possibly referred to as “SEBS”); an ethylene-vinyl acetate copolymer; and an ethylene-alpha-olefin copolymer. Among these, a styrene thermoplastic elastomer that is the thermoplastic block copolymer, such as SIS, SBS, SEPS, and SEBS, is preferably used because of excellent viscosity and aggregating property. Among these, SIS is particularly preferable from the aspects of adhesive capacity to a human skin and a compatibility with other components. A content of SIS is not particularly restricted, however, when a total mass of the adhesive layer is 100 mass %, SIS is preferably 10 mass % to 50 mass %, and more preferably, 10 mass % to 30 mass %. For SIS, a styrene-isoprene-styrene block copolymer that is commercially available can be used, for example, JSR SIS 5002 (JSR Corporation) can be provided as an example.

As the tackiness agent, for example, a rosin resin, a terpene resin, a coumarone-indene resin, a petroleum-based resin (C5 petroleum resin and C9 petroleum resin), an alicyclic petroleum resin, an alicyclic hydrogenated petroleum resin, styrene resin, and a dicyclopentadiene resin are provided as examples. A content of the tackiness agent is not particularly restricted, however, when, for example, the total mass of the adhesive layer is 100 mass %, the tackiness agent can be preferably 15 mass % to 55 mass %, and more preferably, 20 mass % to 50 mass %.

As the softener (plasticizer), a petroleum softener, such as a liquid paraffin; a liquid rubber softener, such as liquid polyisoprene, polybutene, and polyisobutylene; a dibasic acid ester plasticizer, such as phthalic acid ester and adipate; and other plasticizer, such as polyethylene glycol and a citrate, are provided as examples. Among these, the liquid paraffin can be preferably used because of the excellent compatibility with the rubber elastomer and being free from lowering the aggregation. For example, as a commercial product, HICALL (registered trademark, liquid paraffin manufactured by KANEDA Co., Ltd) M series is provided as an example. From the point of viscosity, a content of these softener is, when the total mass of the adhesive layer is 100 mass %, preferably within a range of 25 mass % to 55 mass %, and more preferably 30 mass % to 50 mass %.

Furthermore, the rubber adhesive can further contain, as necessary, an additive that is usually combined in the adhesive layer of a percutaneous absorption drug product, such as a pharmacological agent (active ingredient), an antioxidizing agent (antioxidant agent), a filler, a percutaneous absorption accelerator, pigments, a medicine stabilizer, a medicine dissolution improver, and a medicine dissolution inhibitor. Each of these additives can be used alone, or two or more kinds of these additives can be used in combination.

While the thickness of the adhesive layer is not specifically limited, the thickness is appropriately selectable within a range of, for example, 5 μm to 180 μm, preferably, within a range of 10 μm to 150 μm.

### Carrier Film

In the adhesive tape (tape-typed drug product) used in the disclosure, as described above, the carrier film may be disposed so as to come in close contact with the surface on the opposite side of the adhesive layer side of the backing film. In this case, the support liner described later is attached to the carrier film.

Disposing the carrier film can improve the handleability of the adhesive tape and attachability onto the skin. That is, when the adhesive tape is applied onto the skin, there possibly is a case where the backing film gets wrinkled or the adhesive tape is bent to cause the adhesive layers to adhere to one another. However, the adhesive tape including the carrier film adjacent to the backing film improves a shape retainability of the adhesive tape, thus ensuring a prevention of such problems.

The material of the carrier film is not particularly limited as long as an object to improve the handleability of the adhesive tape is achieved. For example, it is preferred to use various kinds of films made of various kinds of thermoplastic resins, such as polyester, polyurethane, polyethylene, polypropylene, ionomer, polyamide, polyvinyl chloride, polyvinylidene chloride, ethylene vinyl acetate copolymer, thermoplastic polyester, and polytetrafluoroethylene. As the carrier film, one that is in a state of the above-described various kinds of films being laminated to a paper may be used. Among these, it is preferred to use the polyester film from the aspect that the polyester film can make the handleability of the adhesive tape preferable.

The carrier film is preferred to have its thickness thick or be made of a material with a strong stiffness so as to achieve the object to improve the handleability of the adhesive tape. The thickness of the carrier film is normally 10 μm to 500 μm, preferably, 20 μm to 250 μm. When the thickness of the carrier film is less than 10 μm, the backing film and the carrier film of the adhesive tape are not sufficiently brought into close contact. When the thickness of the carrier film exceeds 500 μm, while the adhesion with the backing film of the adhesive tape is sufficient and the operability is improved, the rigidity of the carrier film is excessively increased. Therefore, for example, when the adhesive layer is applied onto the skin after peeling off the release liner at the point of use, the following capability to the skin lacks and the attachability in the curved surface portion and similar portion is insufficient.

For the carrier film, it is preferred to employ one with the tensile strength within a range of 3 N/10 mm to 200 N/10 mm and the tensile elongation within a range of 10% to 1500%. When the tensile strength is less than 3 N/10 mm, the sufficient rigidity is not obtained. The lack of stiffness possibly leads to an insufficient obtainment of an effect of improved handleability of the adhesive tape. On the other hand, when the tensile strength exceeds 200 N/10 mm, while the handleability of the adhesive tape improves, the rigidity is excessively increased to cause insufficient following capability to the skin and attaching force to the curved surface portion when the adhesive tape is applied. Furthermore, when the tensile elongation of the carrier film is less than 10%, the following of the carrier film to the backing film of the adhesive tape is difficult when, for example, applying onto the curved surface portion. When the tensile elongation exceeds 1500%, while the following to the backing film of the adhesive tape is sufficient, when the carrier film extends large compared with the backing film of the adhesive tape, lifting is generated at an interface between the backing film and the carrier film, thereby possibly causing the handleability of the adhesive tape to degrade on the contrary.

In the above-described adhesive tape (tape-typed drug product), one end portion of the carrier film may be left in a state of not contacting and being separated from the surface of the backing film, that is, in the end portion on one side of the carrier film, the non-attached end portion that is not in close contact with the surface of the backing film of the adhesive tape and separated from the backing film may be formed. Disposing the non-attached end portion ensures easy peeling of the carrier film, which is formed by attaching to the support liner, from the backing film of the adhesive tape by grasping and pulling upward the support liner described later and the non-attached end portion together from the side where the non-attached end portion is disposed or, when the support liner has a length that extends long with respect to the outer edge of the carrier film in the side where the non-attached end portion is disposed, by grasping and pulling upward only the support liner from the side where the non-attached end portion is disposed.

The non-attached end portion can be set to a size (area) so as to improve its peel property when the carrier film is peeled off from the backing film. However, the non-attached end portion can be set to a size (area) so as not to be peeled off from the backing film when peeling of the carrier film off from the backing film is not intended, for example, when the adhesive skin patch is stored and when the release liner is firstly peeled off before use of the adhesive skin patch. For example, a proportion of the area of the non-attached end portion is preferred to be 1% to 50% of the whole area of the carrier film.

### Release Liner

The release liner (also referred to as the release layer or the release paper) used in the above-described adhesive skin patch is peeled when the drug product is used and disposed to protect the layer (example: the adhesive layer) that comes in contact with the skin until the time of use and prevent the change of properties.

For the release liner, one that is commonly used in adhesive skin patches in this technical field can be used. For example, examples can be: a plastic film, such as polyester (such as polyethylene terephthalate, polybutylene terephthalate, and polyethylene naphthalate), polypropylene (such as non-stretched and stretched), polyethylene, polyurethane, polyvinyl chloride, and polystyrene; a paper or a synthetic paper, such as a high-quality paper, a glassine paper, a parchment paper, and a kraft paper; a peeling process paper that is, for example, the above-described plastic film, paper or synthetic paper, and a synthetic fiber being coated with a release agent having a release property, such as a silicone resin and a fluorine resin; an aluminum foil; and a sheet that is colorless or colored, such as a laminated process paper variously laminated these film sheets and a laminated peeling process paper that is the laminated process paper coated with the release agent.

While the thickness of these release liners is not specifically limited, it is normally within a range of 10 μm to 1 mm, for example, 20 μm to 500 μm, preferably, 40 μm to 200 μm.

In the disclosure, the size of the release liner is only needed to be longer than the drug product.

That is, the release liner is only needed to be long with respect to at least one direction, such as a longitudinal direction or a short side direction, of the drug product (for example, the backing film of the adhesive tape). Furthermore, the release liner can be long over a whole dimension of the drug product, such as the longitudinal direction and the short side direction of the drug product (for example, the backing film of the adhesive tape). Furthermore, the release liner may be a size that can include a plurality of the drug products.

When the drug product is the adhesive tape, the release liner is only needed to be attached to at least a part of the adhesive layer of the adhesive tape. The release liner may also be attached to a whole surface of the adhesive layer. The release liner is only needed to have a length that can attach the adhesive layer of the adhesive tape, in a further preferable aspect, the release liner can have a length that can attach even the support liner in addition to the adhesive layer of the adhesive tape as described later.

### Support Liner

The above-described support liner used in the disclosure has a laminated structure of at least two layers including the backing film and the adhesive layer disposed on a surface of the backing film.

An existence of the support liner ensures further improved operability of the adhesive skin patch of the disclosure. For example, when the drug product is in a form of the adhesive tape, the support liner can further facilitate peeling of the adhesive tape from the release liner and peeling of the carrier film from the backing film of the adhesive tape. The support liner can further improve the operability when the adhesive layer of the adhesive tape is applied to the applied surface.

The support liner is preferred to be longer than the drug product. For example, when the drug product is in the form of the adhesive tape, the support liner is preferred to be longer than the adhesive tape. In the aspect where the carrier film is further included, the support liner is preferred to be longer than the carrier film, which is formed by being in close contact with the adhesive tape and the backing film of the adhesive tape. In a particularly preferred aspect, the support liner is preferred to have a size formed by attaching to the drug product (for example, the backing film of the adhesive tape or the carrier film) and attaching to the release liner of the adhesive skin patch. In a preferred aspect, the support liner can be one that has a size (length) in which the support liner can attach to the carrier film of the adhesive skin patch in a center of the support liner and the support liner can attach to the release liner of the adhesive skin patch in both ends of the support liner.

Especially, in a preferred aspect, the support liner has the quadrilateral having the long side length of 50.0 mm±5.0 mm and the short side length of 5.0 mm or more and 37.5 mm or less.

### Backing Film

As the backing film of the above-described support liner, the tape-typed drug product as one form of the above-described drug product: one that is identical to the backing film provided as an example in the adhesive tape can be employed. Among these, the polyethylene film, a polyolefin nonwoven fabric, a cyclic polyolefin film, and the polyester film are preferred from the aspect of making the handleability of the adhesive skin patch satisfactory, in particular, the polyethylene film and the polyolefin nonwoven fabric are preferred.

The backing film can be set to have, for example, its thickness within a range of 25 μm to 500 μm considering the physical property, such as the tensile elongation, the tensile strength, and the workability.

The backing film is preferred to have an appropriate flexibility so as to improve the handleability of the adhesive skin patch and the drug product (the adhesive tape), for example, 60 mm or more of bending resistance is preferred.

On a surface on the side where the adhesive layer of the backing film is not disposed, usage instructions of the adhesive skin patch of the disclosure, for example, peeling procedures and peeling instructions of the release liner, kinds (kinds of combined active ingredients) of the drug product (adhesive tape), can be specified by means of printing and similar means.

### Adhesive Layer

As the adhesive layer of the above-described support liner, the above-described tape-typed drug product: one that is identical to the adhesive layer in the adhesive tape can be employed. For example, the acrylic adhesive, the rubber adhesive, the urethane adhesive, and the silicone adhesive can be provided as examples. It is preferable to use the rubber adhesive from the aspect of the compatibility with the compound components.

While the thickness of the adhesive layer of the support liner is not specifically limited, the thickness is appropriately selectable within a range of, for example, 5 μm to 180 μm, preferably, 10 μm to 150 μm.

### WORKING EXAMPLE

The following indicates and further specifically describes working examples of the disclosure, however, the disclosure is not limited to these, and various kinds of applications are allowed within the range not departing the technical idea of the disclosure.

### Working Example 1: Adhesive Skin Patch in which Support Liner is Attached

Following the following procedure, an adhesive skin patch in a form of an adhesive tape in which a drug product includes a carrier film was manufactured.

### Manufacturing Method of Support Liner Material

A coating liquid to form an adhesive layer with a solid content of 40 mass % was prepared by mixing 100 pts. mass of a styrene-isoprene-styrene block copolymer (JSR SIS5002 manufactured by JSR Corporation), 62.5 pts. mass of a terpene resin (YS resin manufactured by YASUHARA CHEMICAL CO., LTD.) as a tackifying resin, and 87.5 pts. mass of a liquid paraffin (HICALL (registered trademark) M-352 manufactured by KANEDA Co., Ltd) as a softener, and dissolving them in a mixed solvent of toluene/hexane=8/2 (mass ratio).

This coating liquid was applied using a bar coater over one surface of a release liner (a silicone processed polyethylene terephthalate film, 25 μm of thickness) on which a release layer was to be formed. The coating liquid was applied so as to have a thickness of 25 μm after drying. Next, the coating liquid was dried. After a laminated body constituted of the release layer and the adhesive layer was obtained, a backing film constituted of a polyethylene film with 80 μm of thickness was laminated onto the adhesive layer to provide a support liner material.

### Preparation of Coating Liquid to Form Adhesive Layer of Adhesive Tape

A coating liquid to form an adhesive layer with a solid content of 57 mass % was prepared by mixing 100 pts.mass of a styrene-isoprene-styrene block copolymer (JSR SIS5002 manufactured by JSR Corporation), 250 pts.mass of a terpene resin (YS resin manufactured by YASUHARA CHEMICAL CO., LTD.) as the tackifying resin, and 350 pts.mass of a liquid paraffin (HICALL (registered trademark) M-352 manufactured by KANEDA Co., Ltd) as the softener, and dissolving them in a mixed solvent of toluene/acetone=7/3 (mass ratio).

Manufacturing Adhesive Skin Patch in which Support Liner is Attached

First, the above-described coating liquid to form the adhesive layer of the adhesive tape was applied using the bar coater over one surface of a release liner (a silicone processed polyethylene terephthalate film, 75 μm of thickness). The coating liquid was applied so as to have a thickness of 20 μm after drying. Next, the coating liquid was dried, and a laminated body A constituted of the release liner and the adhesive layer was obtained.

Subsequently, by extruding a molten resin material (polyethylene) that constitutes a backing film of the adhesive tape to the carrier film made of the polyethylene terephthalate, a laminated body B constituted of a backing film made of the polyethylene film with 15 μm of thickness and the carrier film made of the polyethylene terephthalate with 25 μm of thickness was obtained.

The laminated body A and the laminated body B were laminated such that the above-described adhesive layer of the laminated body A and the above-described backing film of the laminated body B overlapped to provide a laminated body including the release liner and a drug product (formed by including the adhesive layer, the backing film, and the carrier film in this order).

Then, a non-attached end portion was formed by partly peeling the carrier film and the backing film made of the polyethylene film at an end portion of the laminated body A. The non-attached end portion was formed to have an area less than 50% of a whole area of the carrier film when the carrier film being as the adhesive tape, which will be described later.

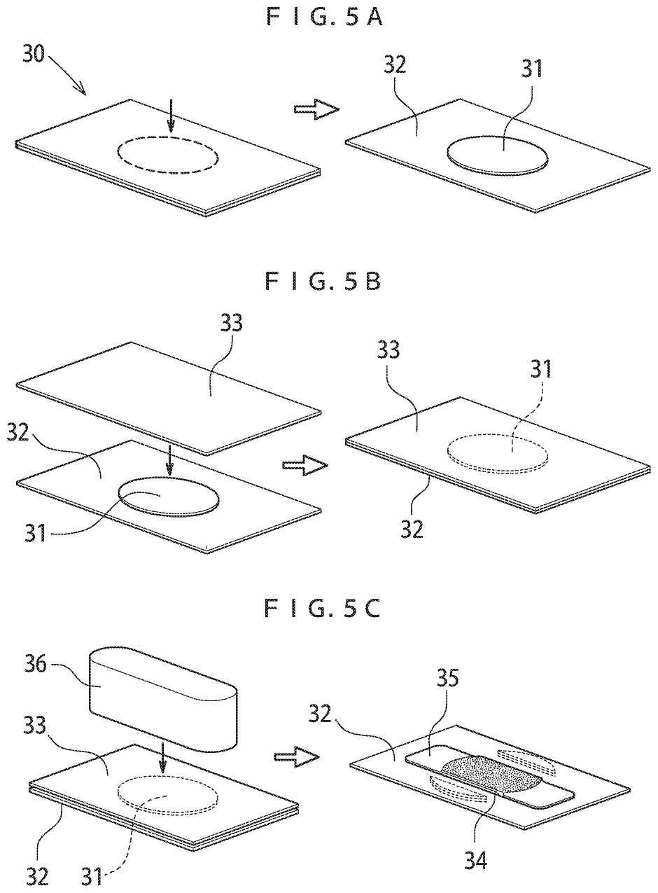

In the following, an adhesive skin patch in which a support liner is attached was manufactured by procedures illustrated in FIGS. 5A to 5C. In FIGS. 5A to 5C, a detailed laminated structure of the drug product (the structure formed by including the adhesive layer, the backing film, and the carrier film in this order) and the non-attached end portion of the carrier film are omitted from illustration.

1. Step 1 of Punching Out Drug Product into Elliptical Shape (Shape Including Elliptical Arc) (FIG. 5A)

In a laminated body 30 including the release liner and the drug product obtained by laminating the above-described laminated body A and laminated body B, only the drug product was half-cut (a release liner 32 was not cut) such that the non-attached end portion was included and an elliptical shape has an ellipse major axis L₁ of 30.0 mm and an ellipse minor axis M₁ of 24.6 mm to obtain a drug product 31 in the elliptical shape that was attached onto the release liner 32.

### 2. Step 2 of Attaching Support Liner Material to Whole Surface of Drug Product (FIG. 5B)

The release liner attached to the support liner material that was made above was peeled. Then, a support liner material 33 was laminated over the drug product 31 (what attached actually was the carrier film (adhesive tape)) in the elliptical shape and a whole surface of the release liner 32 around portions where the drug product 31 was attached (a portion where the tape drug product was not attached) so as to cover a whole surface of the drug product cut into the elliptical shape having the ellipse major axis L₁ of 30.0 mm and the ellipse minor axis M₁ of 24.6 mm.

3. Step 3 of Obtaining Support Liner on which Quadrilateral Drug Product is Attached (FIG. 5C)

Then, only the support liner material 33 and the drug product 31 formed by having the support liner material 33 attached were half-cut (the release liner 32 was not cut) into a shape of the support liner (a metallic mold 36) (a quadrilateral with a long side L₂ of 50.0 mm and a short side M₂ of 14.0 mm). When being half-cut, the support liner material (and the adhesive tape formed by attaching to the support liner material) was cut (displacement of punching position: 0.0 mm, a drug product area S₀) such that a center axis in the long side direction of the drug product in the elliptical shape and a center axis in the long side direction of the quadrilateral of the support liner after the half-cut became parallel and a center axis in the short side direction of the drug product in the elliptical shape and a center axis in the short side direction of the quadrilateral of the support liner after the half-cut were on an identical straight line and a center of gravity of the drug product in the elliptical shape and a center of gravity of the quadrilateral of the support liner after the half-cut overlapped. Similarly, the support liner material (and the adhesive tape formed by attaching to support liner material) was half-cut (displacement of punching position: 1.0 mm, a drug product area S₁) shifted such that a distance between the center of gravity of the drug product in the elliptical shape and the center of gravity of the quadrilateral of the support liner after the half-cut became 1.0 mm in a central axis direction of both the short side directions.

From above-mentioned steps, the adhesive skin patches (two kinds of the adhesive skin patches having the displacements of punching positions of 0.0 mm and 1.0 mm) in which the support liners were attached according to Working Example 1 were obtained. The adhesive skin patch includes the quadrilateral drug product with the two long sides being the straight lines of approximately equal lengths in parallel with one another and the two short sides being the pair of elliptical arcs that convex outward having lengths equal to one another. The drug product has the longest interval between the two short sides of 30.0 mm and the interval between the two long sides of 14.0 mm.

### Working Examples 2 to 14

The adhesive skin patches (two kinds of the adhesive skin patches having the displacements of punching positions of 0.0 mm and 1.0 mm for the respective working examples) in which the support liners of Working Examples 2 to 14 were attached were obtained similarly to the procedures of Working Example 1 except that the ellipse major axis L₁, the ellipse minor axis M₁, and the short side M₂ of the support liner were set to values indicated in Table 1.

### Reference Examples 1 to 4

The adhesive skin patches (two kinds of the adhesive skin patches having the displacements of punching positions of 0.0 mm and 1.0 mm for the respective working examples) in which the support liners of Reference Examples 1 to 4 were attached were obtained similarly to the procedures of Working Example 1 except that the ellipse major axis L₁, the ellipse minor axis M₁, and the short side M₂ of the support liner were set to values indicated in Table 2.

### Reference Example 5

With the following procedures (based on the procedures of Working Example 1 except for the procedures otherwise specified), an adhesive skin patch in which the support liner of Reference Example 5 was attached was obtained. The adhesive skin patch has the distance between the long sides of the support liner (that is, the short side of the support liner) larger by 2.0 mm than the distance between the long sides of the drug product (that is, the short side of the drug product).

That is, after half-cutting only the drug product into the elliptical shape at “1. Step 1 of Punching Out Drug Product into Elliptical Shape (Shape Including Elliptical Arc),” using the metallic mold 36 (quadrilateral shape with the long side L₂ of 50.0 mm and the short side M₂ of 14.0 mm) used in Working Example 1, only the drug product was further half-cut. When the half-cut was performed using the metallic mold, the half-cut was performed by adjusting a position of the metallic mold 36 such that the center axis in the long side direction of the drug product in the elliptical shape (before the half-cut) and the center axis in the long side direction of the drug product after the half-cut became parallel and the center axis in the short side direction of the drug product in the elliptical shape (before the half-cut) and the center axis in the short side direction of the drug product after the half-cut were in an identical straight line and the center of gravity of the drug product in the elliptical shape (before the half-cut) and the center of gravity of the drug product after the half-cut overlapped.

Then, at “3. Step 3 of Obtaining Support Liner On Which Quadrilateral Drug Product Is Attached,” a quadrilateral metallic mold with the long side L₂ of 50.0 mm and the short side M₂ of 16.0 mm was used as the metallic mold 36 when the support liner material 33 was half-cut into the shape of the support liner. At this time, the support liner material was cut (displacement of punching position: 0.0 mm, the drug product area S₀) such that the center axis in the long side direction of the drug product after the half-cut at the above-described Step 1 and the center axis in the long side direction of the quadrilateral of the support liner after the half-cut at the Step 3 became parallel, and the center axis in the short side direction of the drug product after the half-cut at the above-described Step 1 and the center axis in the short side direction of the quadrilateral of the support liner after the half-cut at the Step 3 were in the identical straight line and the center of gravity of the drug product after the half-cut at the above-described Step 1 and the center of gravity of the support liner after the half-cut at the Step 3 overlapped.

Thus, the adhesive skin patch according to Reference Example 5 that has the short side of the support liner larger by 2.0 mm than the short side of the drug product, that is, formed by the respective long sides of the support liner being positioned at outer side by 1.0 mm each from the respective long sides of the drug product was obtained.

### Handleability

Evaluations of the handleabilities (easiness of application) of the adhesive skin patches according to Working Example 1 as the product of the disclosure and Reference Example 5 were evaluated by the following method.

That is, the adhesive skin patch according to Working Example 1 (the displacement of the punching position is 0.0 mm and the short side of the support liner is identical to the short side of the drug product) and the adhesive skin patch according to Reference Example 5 (the displacement of the punching position is 0.0 mm and the short side of the support liner is larger by 2.0 mm than the short side of the drug product) were applied onto eyelids of respective twelve examinees of adult men and women. At this time, the handleabilities were evaluated according to the following scoring in order to evaluate differences of the easiness of application due to the sizes of the support liner. The average score of the evaluations of the examinees was calculated to set the average score of 4.0 or more to excellent (extremely excellent in the handleability of the product), 3.5 or more and less than 4.0 to good (excellent in the handleability of the product), 3 or more and less than 3.5 to fair (lacking in the handleability of the product), and less than 3 to poor (inadequate in the handleability of the product).

### <Easiness of Application Due to Size of Support Liner>

5: Just right

3: Slightly large

1: Too large and problematic

For the adhesive skin patches obtained by Working Examples 1 to 14 and Reference Examples 1 to 4, the drug product areas S₀ and S₁ (mm²) are indicated in Tables 1 and 2. The drug product areas S₀ and S₁ (mm²) are when the displacements of the punching positions (the distances between the centers of gravities of the drug products in the elliptical shapes and the centers of gravities of the support liners after the half-cut) at the time of half-cutting into the support liner shape are 0.0 mm and 1.0 mm. In Tables 1 and 2, area change ratios (%) indicate changing ratios (%) of the drug product areas S₁ when the displacements of the punching positions are 1.0 mm with respect to the drug product areas S₀ when the displacements of the punching positions are 0.0 mm (the centers of gravities of the drug products in the elliptical shapes and the centers of gravities of the support liners after the half-cut overlap). In Tables 1 and 2, the support liner short sides M₂/the ellipse major axes L₁ correspond to the short sides/the long sides of the quadrilateral drug products in the adhesive skin patches of the respective examples.

For the adhesive skin patches obtained by Working Example 1 and Reference Example 5 (both the displacements of the punching positions: 0.0 mm), Table 3 indicates results of evaluation of the easiness of application.

| | Working Examples | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| Ellipse Major | 30.0 | 30.0 | 30.0 | 30.0 | 20.0 | 40.0 | 45.0 | 20.0 | 25.0 | 30.0 | 20.0 | 30.0 | 40.0 | 50.0 |
| Axis L₁ [mm] | | | | | | | | | | | | | | |
| Ellipse Minor | 24.6 | 25.0 | 20.0 | 19.2 | 16.5 | 22.7 | 24.8 | 19.2 | 21.8 | 24.8 | 14.8 | 15.5 | 16.5 | 17.7 |
| Axis M₁ [mm] | | | | | | | | | | | | | | |
| Support Liner | 14.0 | 15.0 | 15.0 | 15.0 | 10.0 | 20.0 | 22.5 | 15.0 | 18.8 | 22.5 | 5.0 | 7.5 | 10.0 | 12.5 |
| Short Side | | | | | | | | | | | | | | |
| M₂ [mm] | | | | | | | | | | | | | | |
| M₂/L₁ [−] | 0.47 | 0.50 | 0.50 | 0.50 | 0.50 | 0.50 | 0.50 | 0.75 | 0.75 | 0.75 | 0.25 | 0.25 | 0.25 | 0.25 |
| Drug Product | 396.1 | 421.3 | 403.2 | 398.7 | 186.9 | 679.2 | 846.7 | 265.8 | 401.6 | 564.5 | 98.1 | 215.9 | 373.9 | 568.4 |
| Area S₀ [mm²] | | | | | | | | | | | | | | |
| Drug Product | 394.4 | 419.5 | 399.8 | 394.7 | 185.1 | 672.4 | 838.2 | 263.1 | 397.6 | 558.9 | 97.1 | 213.7 | 370.1 | 562.7 |
| Area S₁ [mm²] | | | | | | | | | | | | | | |
| Area Change | 0.4 | 0.4 | 0.8 | 1.0 | 1.0 | 1.0 | 1.0 | 1.0 | 1.0 | 1.0 | 1.0 | 1.0 | 1.0 | 1.0 |
| Ratio [%] | | | | | | | | | | | | | | |

TABLE 2

| | Reference Examples | | | |
|---|---|---|---|---|
| | 1 | 2 | 3 | 4 |
| Ellipse Major Axis L₁ [mm] | 19.0 | 20.0 | 20.0 | 30.0 |
| Ellipse Minor Axis M₁ [mm] | 18.0 | 14.0 | 14.0 | 14.0 |
| Support Liner Short Side M₂ [mm] | 14.3 | 10.0 | 5.0 | 7.5 |
| M₂/L₁ [—] | 0.75 | 0.50 | 0.25 | 0.25 |
| Drug Product Area S₀ [mm²] | 238.9 | 181.4 | 97.8 | 213.7 |
| Drug Product Area S₁ [mm²] | 236.1 | 178.4 | 96.7 | 211.0 |
| Area change ratio [%] | 1.2 | 1.7 | 1.1 | 1.3 |

TABLE 3

| | Working Example 1 | Reference Example 5 |
|---|---|---|
| Easiness of Application | Excellent | Fair |

As Tables 1 and 2 indicate, in Working Examples 1 to 14, even when the displacements of the punching positions were 1.0 mm, the change ratios of the drug product areas from 0.0 mm of the displacements of the punching positions were 1.0% or less. Therefore, the changes of the drug product areas could be made small.

On the other hand, Reference Example 1 that is outside of the numerical value range of the ellipse major axis of the elliptical arc and Reference Examples 2 to 4 that are outside of the numerical value range of the ellipse minor axis of the elliptical arc specified by the disclosure all resulted in having the area change ratios of the drug product areas exceeding 1.0% and resulted to show that the changes of the drug product areas were large compared with Working Examples.

Furthermore, as Table 3 indicates, the handleability of the adhesive skin patch according to Working Example 1 was excellent compared with Reference Example 5.

1. An adhesive skin patch in which a support liner is attached, the adhesive skin patch comprising at least:
a drug product in a form of a strip; and
a release liner longer than the drug product, wherein
the support liner includes a backing film and an adhesive layer disposed on a surface of the backing film, the support liner being attached to the drug product,
the drug product is a quadrilateral drug product with two long sides and two short sides, the two long sides being straight lines of approximately equal lengths in parallel with one another, the two short sides being a pair of elliptical arcs that are convex outward having lengths equal to one another,
a longest interval between the two short sides of the drug product is 20.0 mm or more and 50.0 mm or less, and an interval between the two long sides of the drug product is 5.0 mm or more and 37.5 mm or less, and
the elliptical arc is an arc of an ellipse (however, not a true circle arc) having: an ellipse major axis of 20.0 mm or more and 50.0 mm or less; and an ellipse minor axis of 14.8 mm or more and less than 50.0 mm, and the minor axis of at least 2.0 mm wider than the interval between the two long sides of the drug product.

2. The adhesive skin patch according to claim 1, wherein
in the quadrilateral drug product,
the longest interval between the two short sides is 28.0 mm or more and 32.0 mm or less, and the interval between the two long sides is 12.0 mm or more and 16.0 mm or less, and
the elliptical arc is an arc of an ellipse having the ellipse major axis of 28.0 mm or more and 32.0 mm or less and the ellipse minor axis of 23.0 mm or more and 27.0 mm or less.

3. The adhesive skin patch according to claim 1, wherein:
the drug product comprises an adhesive tape that is a tape-typed drug product in a form of a strip, the adhesive tape including a backing film and an adhesive layer disposed on one surface of the backing film; and
the release liner is attached to the adhesive layer of the adhesive tape, the release liner being longer than the backing film of the adhesive tape, and
the support liner is attached to the backing film of the adhesive tape.

4. The adhesive skin patch according to claim 3, wherein
in the adhesive skin patch,
the adhesive tape further includes a carrier film that is brought into close contact with a surface on a side of the backing film of the adhesive tape opposite to the side on which the adhesive layer of the adhesive tape is disposed, and
the support liner is attached to the carrier film.

5. The adhesive skin patch according to claim 4,
in the adhesive skin patch,
the carrier film includes one end portion out of close contact with a surface of the backing film of the adhesive tape and separated from the backing film to form a non-attached end portion.

6. The adhesive skin patch according to claim 1, wherein the adhesive skin patch is an adhesive skin patch for an eyelid.

7. A manufacturing method of an adhesive skin patch in which a support liner is attached, the adhesive skin patch comprising at least:
a drug product in a form of a strip; and
a release liner longer than the drug product, wherein
the support liner includes a backing film and an adhesive layer disposed on a surface of the backing film, the support liner being attached to the drug product,
the drug product is a quadrilateral drug product with two long sides and two short sides, the two long sides being straight lines of approximately equal lengths in parallel with one another, and the two short sides being a pair of elliptical arcs that are convex outward having lengths equal to one another, and
a longest interval between the two short sides of the drug product is 20.0 mm or more and 50.0 mm or less, and an interval between the two long sides of the drug product is 5.0 mm or more and 37.5 mm or less, wherein
the manufacturing method comprises:
a step of punching out the drug product into a shape including the elliptical arc having: an ellipse major axis of 20.0 mm or more and 50.0 mm or less; and an ellipse minor axis of 14.8 mm or more and less than 50.0 mm, and the minor axis of at least 2.0 mm wider than the interval between the two long sides of the drug product;
a step of attaching a support liner material over a whole surface of the drug product punched out into the shape including the elliptical arc; and
a step of obtaining the support liner on which the quadrilateral drug product is attached by punching out the support liner material together with the drug product formed by having the support liner material attached.

8. The manufacturing method according to claim 7, wherein
in the quadrilateral drug product,
the longest interval between the two short sides is 28.0 mm or more and 32.0 mm or less, and the interval between the two long sides is 12.0 mm or more and 16.0 mm or less, and
the step of punching out the drug product into the shape including the elliptical arc is a step of punching out the drug product into the shape including the elliptical arc that is an arc of an ellipse having the ellipse major axis of 28.0 mm or more and 32.0 mm or less and the ellipse minor axis of 23.0 mm or more and 27.0 mm or less.

9. The manufacturing method according to claim 7, wherein
the step of obtaining the support liner is a step of punching out the support liner material into a quadrilateral having a long side length of 50.0 mm±5.0 mm and a short side length of 5.0 mm or more and 37.5 mm or less.

10. The manufacturing method according to claim 7, wherein
the step of obtaining the support liner is a step of punching out the support liner material together with the drug product formed by having the support liner material attached such that:
a center axis in a long side direction of the drug product in the shape including the elliptical arc and a center axis in a long side direction of the quadrilateral of the support liner become parallel;
a center axis in a short side direction of the drug product in the shape including the elliptical arc and a center axis in a short side direction of the quadrilateral of the support liner are on an identical straight line; and
a distance between a center (center of gravity) in the short side direction of the drug product in the shape including the elliptical arc and a center (center of gravity) in the short side direction of the quadrilateral of the support liner is 1.0 mm or less.
